(19) United States
(12) Patent Application Publication
(10) Pub. No.: US 2023/0314314 A1
(43) Pub. Date: Oct. 5, 2023

(54) OPTICAL SYSTEM AND METHOD TO IDENTIFY PLASTIC

(71) Applicant: WOODS HOLE OCEANOGRAPHIC INSTITUTION, Woods Hole, MA (US)

(72) Inventor: Anna MICHEL, Woods Hole, MA (US)

(73) Assignee: WOODS HOLE OCEANOGRAPHIC INSTITUTION, Woods Hole, MA (US)

(21) Appl. No.: 17/924,329

(22) PCT Filed: May 11, 2021
(86) PCT No.: PCT/US2021/031733

§ 371 Date: Nov. 9, 2022

Related U.S. Application Data
(60) Provisional application No. 63/022,722, filed on May 11, 2020.

(51) Int. Cl. G01N 21/3563; G02B 27/00; G01N 33/44

(52) U.S. Cl. CPC G01N 21/3563; G01N 33/442; G02B 27/0006; G01N 2201/0636

(57) ABSTRACT

A system for the identification of sample types and method of using same. The system and method including an optical mechanism, and a detector. The system accepts a sample, directs input light from the optical mechanism onto that sample, producing output light that is received by the detector. The detector produces a sample-derived spectrum for classification. The present disclosure provides for a classification method to classify spectrum into one of many sample types, preferably plastic types.

### CROSS-REFERENCE TO RELATED APPLICATION

This application claims benefit to U.S. Provisional Pat. Application No. 63/022,722, filed May 11, 2020, the disclosure of which is hereby incorporated by reference in its entirety.

### FIELD OF THE DISCLOSURE

This disclosure relates to the identification of plastic compositions. More particularly this disclosure relates to the identification of different chemical compounds, typically polymers that make up plastics. In some embodiments, this disclosure relates to the identification of plastic types in small plastic particles and in some embodiments, in microplastics.

### BACKGROUND OF THE DISCLOSURE

The robust and rapid identification of plastic type is needed for environmental applications ranging from recycling facilities to understanding sources and sinks of plastics in the environment. For example, to understand plastic fate and transport in the environment, it is important to be able to robustly classify plastics found in locations ranging from land (i.e., a terrestrial environment) to the deep sea (i.e., an aquatic environment). Although hundreds of types of plastics exist with added complexity due to fillers, additives, and colorants; plastics are often identified by their recycling codes (and can be thought of in broad plastic types): #1 polyethylene terephthalate (PET), #2 high density polyethylene (HDPE), #3 polyvinyl chloride (PVC), #4 low density polyethylene (LDPE), #5 polypropylene (PP), #6 polystyrene (PS), and #7 other plastics.

Optical approaches for plastics classification allow samples to be identified in both stand-off configurations and in a non-destructive manner, with no damage to a sample. A range of optical approaches have been utilized for plastics classification, including attenuated total reflectance - Fourier transform infrared spectroscopy (ATR-FTIR), laser induced breakdown spectroscopy (LIBS), near-infrared reflectance spectroscopy (NIR), and Raman spectroscopy. Hybrid approaches, such as combining Raman and LIBS techniques, have also been applied to plastics identification. There are challenges to some of these approaches; for example, NIR spectroscopy is limited for plastics identification, as the wavelengths cannot be used to identify black or dark (e.g. dark grey, or otherwise opaque) plastics due to their low reflectance in the NIR spectral range. In the NIR region, other materials such as carbon black and soot both absorb completely. For polymer sorting, Fourier transform infrared (FTIR) spectroscopy is typically too slow. To address these limitations, there is a need for a wider range of optical approaches for plastic identification be explored.

Mid-infrared (MIR) wavelengths are of particular interest for plastics identification, in particular, MIR Quantum cascade lasers (QCLs) are compact and can be made both high power and widely tunable. QCLs have been demonstrated to have applications ranging from detection of trace gases, explosives, and medically relevant compounds such as glucose, lactate and triglycerides. The ability to be widely tunable makes them a viable source for covering a large spectral range and for measuring broadband absorbers. The compact design of the QCL and its ability to be used in stand-off or remote operation make this solution a viable source for implementation in small, field portable sensors. Specular reflectance spectroscopy is a powerful and simple approach that requires only optical access to a sample. Such an approach reduces the possibility of sample cross-contamination. For example, other MIR wavelength techniques such as FTIR and ATR-FTIR, require physical contact with the sample, which could result in cross-contamination if the sample, residues, or biofilms stick to the contacting crystal. Disclosed herein is a novel demonstration of QCL-based reflectance spectroscopy coupled with a classifier technique, linear discrimination, for the accurate identification of plastic samples.

### SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The identification of plastic type is important for environmental applications ranging from recycling to understanding the fate of plastics in marine, atmospheric, and terrestrial environments. Infrared reflectance spectroscopy is a powerful approach for plastics identification, requiring only optical access to a sample. The use of visible and near-infrared wavelengths for plastics identification are limiting as dark colored plastics absorb at these wavelengths, producing no reflectance spectra. The use of mid-infrared wavelengths instead enables dark plastics to be identified. Herein is demonstrated in one embodiment is the capability to utilize a pulsed, widely-tunable (5.59 - 7.41 µm) mid-infrared quantum cascade laser, as the source for reflectance spectroscopy, for the rapid and robust identification of plastics. Through the application of linear discriminant analysis to the resulting spectral data set, this disclosure correctly classify five plastic types polyethylene terephthalate (PET), high density polyethylene (HDPE), low density polyethylene (LDPE), polypropylene (PP), and polystyrene (PS), with a 97% accuracy rate.

In some embodiments, this disclosure features a system for the identification and classification of plastic types of a sample, the system comprising a quantum cascade laser configured to produce input light capable of impinging a sample and thereby creating output light from the input light impinging the sample; a detector to receive the output light, producing a spectrum; and a processor configured to classify at least one property of the spectrum using a classification model. In one embodiment, the classification model maps a portion of the spectrum into a first category and where the first category is a type of plastic. In some of these embodiments, a second or more categories are available for classification. In some of these embodiments, the categories are types of plastic.

In one embodiment, the system also has a space between the quantum cascade laser and the sample, where the input light travels through the space. In some embodiments, the output light is created after the input light transmits a single time through the sample and the input light does not reflect through the sample. In some of these embodiments, the output light does not reflect through the sample. In some embodiments, the system also has a mirror and the output light transmits a first time through the sample, reflects off the mirror and transmits a second time through the sample. In some embodiments, the system also has a housing, an interface, and a power source, where the housing provides protection from environmental factors to the quantum cascade laser, detector, processor and power source. In some of these embodiments, the system is portable. In some of these embodiments, the system is configured to be picked up and moved by a single person.

In one embodiment, the system also has a light modifier configured to modify the output light. In another embodiment, the system also has a light modifier configured to modify the input light. In some embodiments, the classification model is also configured to normalize and transform the spectrum. In some of these embodiments, the classification model is supplied with a training set and a test set; and the classification model is further configured to apply an analyzer routine on the test set. In some of these embodiments, the analyzer routine is applied to training set.

6. In some embodiments, this disclosure features a model to classify a type of plastic, the method including the steps of (a) selecting a system comprising a quantum cascade laser, a sample holder, and a detector; the quantum cascade laser configured to produce input light for impinging a sample; (b) placing a sample onto the sample holder; (c) directing input light onto the sample, thereby producing output light; (d) capturing the output light by the detector thereby producing a machine-readable spectrum; and (e) analyzing the spectrum with a classification model to classify at least one property of the spectrum, thereby producing a classification result. In some methods, the classification model classifies the at least one property of the spectrum into one category of a plurality of categories, and where that one category of the plurality of categories is a type of plastic. Some methods also have the steps of (f) applying a normalization routine to the spectrum, producing a normalized spectrum and (g) applying a transformation routine to the normalized spectrum, producing a transformed spectrum; and where the spectrum in step (e) analyzed with the classification model is the transformed spectrum. In one of these embodiments, the method further includes the step of (h) applying a smoothing routine to said transformed spectrum, producing a smoothed spectrum, and where the spectrum in step (e) analyzed with the classification model is the smooth spectrum. In some of these methods, the method includes the steps of (i) repeating all of steps (b) through (h) the repetitions producing a plurality of smoothed spectrum; (j) forming a test set from at least a portion of the plurality of smoothed spectrum; and (k) applying an analyzer routine to the test set to produce, in part, the classification result.

7. In some of the above methods, the output light is created after the input light transmits only a single time through the sample, without a reflection. In some of the above methods, the system further comprises a mirror, and the output light transmits a first time through the sample, reflects off the mirror and transmits a second time through the sample. In some of these embodiments, the second output light transmission reflects at an angle other than 180 degrees from the sample. In some of the above embodiments, the system also has a housing, an interface, and a power source, the housing providing a protection from environmental factors and the quantum cascade laser, the detector, the processor, and the power source are located within the housing. In some of these methods, the housing is configured to be manually moved by a user. In some of these methods, the method further includes the step of (1) manually moving the system by a user. In some of these methods, the method further includes the step of lifting the system by a user.

### DEFINITIONS

The term “plastic” as used herein uses its normal dictionary meaning of a synthetic material made from a wide range of polymers, that are moldable into a shape while soft, and are settable into a rigid or partially elastic form after hardening. The term plastic encompasses many different polymers, including but not limited to polypropylene (PP), polyvinyl chloride (PVC), polystyrene (PS), nylon, polyethylene, High-Density Polyethylene (HDPE), and polyethylene terephthalate (PET).

The term “input light” as used herein refers to the electromagnetic radiation produced by the optical mechanism 101 before it impacted with sample 104. The term “output light” as used in this disclosure refers to electromagnetic radiation that has impacted with the sample and is directed to detector 102. And the term “light path” as used herein, includes all of the radiation emitted and detected by a system, including the input light and the output light.

### BRIEF DESCRIPTION OF THE DRAWINGS

In what follows, preferred embodiments of the disclosure are explained in more detail with reference to the drawings, in which:

FIGS. 1A and 1B are two schematic overview diagrams of two systems according to two embodiments of the disclosure.

FIGS. 2A-2E are representative spectra (smoothed with a moving average of 150 data points and shown normalized on a 0 to 1 scale) from the embodiment represented in the Example of PET (sample PET02) in FIG. 2A, HDPE (sample HDPE28) in FIG. 2B, PP (sample PP 22) in FIG. 2C, LDPE (sample LDPE17) in FIG. 2D, and PS (sample PS05) in FIG. 2E. Peaks corresponding to plastic type are labeled. Peaks are shown in cm⁻¹.

FIG. 3 shows the variability between spectra of 5 different PET samples (shown normalized on a 0 to 1 scale). (Front to back: PET19, PET01, PET13, PET21, PET07). The characteristic PET peaks at 1736 cm⁻¹ and 1411 cm⁻¹ remain in each sample, and increased variability in the 1697-1550 cm⁻¹ region is not indicative of plastic type and not used for analysis.

FIGS. 4A-4C are comparison of spectra of a black and a white HDPE plastic sample. FIG. 4A is a photograph of a black HDPE sample. FIG. 4B is a photograph of a white HDPE sample. FIG. 4C is a characteristic spectral peaks at 1473 cm⁻¹ and 1463 cm⁻¹ of a HDPE sample. Spectra smoothed by 150 point moving average and shown normalized on a 0 to 1 scale. The spectrum illustrated with a black line represents a black HDPE sample. The spectrum illustrated with a grey line represents a white HDPE sample.

FIGS. 5A-5E show ATR-FTIR representative spectra (shown normalized on a 0 to 1 scale) from the Example of a PET (sample PET02) in FIG. 5A, in FIG. 5B a HDPE sample (#HDPE28), in FIG. 5C a PP sample (#PP 22), in FIG. 5D a LDPE sample (# LDPE17), and in FIG. 5E a PS sample (#PS05). Characteristic peaks corresponding to each plastic type are labeled. Peaks are shown in cm⁻¹.

FIGS. 6A-6E show Reflectance FTIR representative spectra (smoothed with a moving average of 10 data points and shown normalized) of FIG. 6A: PET (sample PET02), FIG. 6B: HDPE (sample HDPE28), FIG. 6C: PP (sample PP 22), FIG. 6D: LDPE (sample LDPE17), FIG. 6E: PS (sample PS05). Characteristic peaks corresponding to plastic type are labeled. Peaks are shown in cm⁻¹.

FIG. 7 is a representative flow diagram illustrating the disclosed method with a system according to one embodiment.

FIG. 8 is a representative flow diagram illustrating one method of use according to one embodiment of the present disclosure.

FIG. 9 is a representative flow diagram illustrating one classification model according to one embodiment of the present disclosure.

FIG. 10 is a representation of one portable embodiment.

### DETAILED DESCRIPTION

### Overview

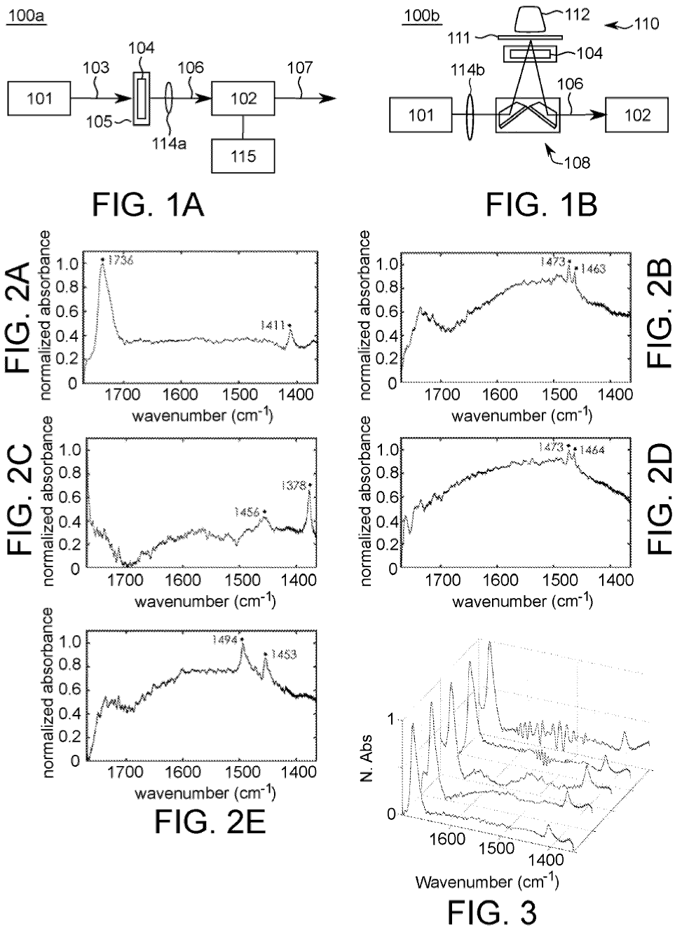

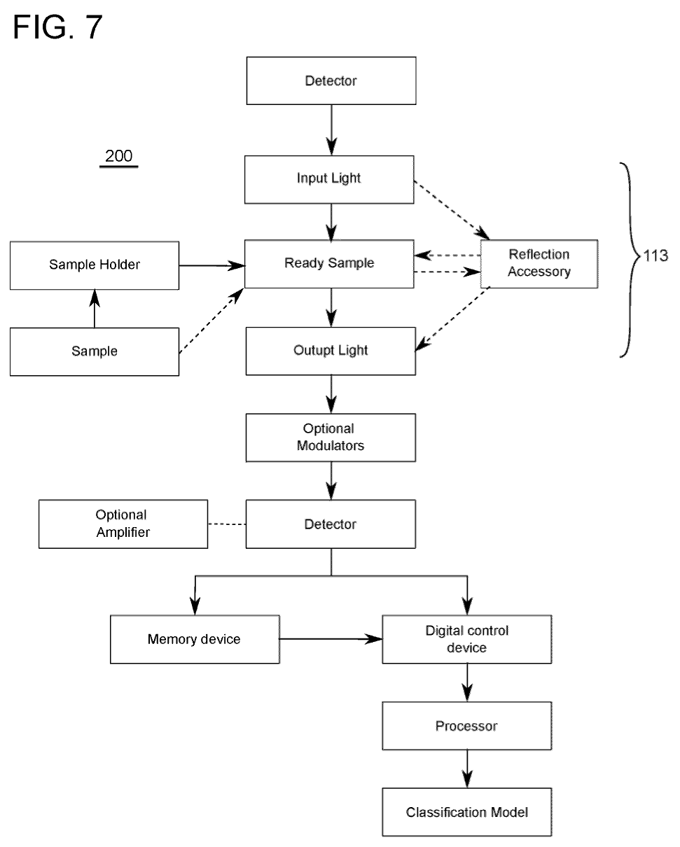

A system 100 and method 200 are provided as improved technical solutions for the identification of plastic materials, including small or micro-sized plastic samples 104. The disclosure may be accomplished by a system 100 having, at a minimum, an optical mechanism 101 and a detector 102. The optical mechanism 101 directs electromagnetic radiation, referred herein as input light 103, onto a sample 104. Samples 104 are accepted into the system 100, preferably with an optional sample holder 105. The sample 104 is illuminated with input light 103, producing a light-spectrum, referred herein as output light 106. The output light 106 is captured by detector 102, producing a machine-readable spectrum of electromagnetic radiation, referred herein as a spectrum 107. The totality of the input light 103 and output light 106 is referred together as the light path 113, illustrated by bracket 113 in FIG. 7. The present disclosure may be accomplished in a transmissive embodiment, as illustrated in FIG. 1A or in a reflective embodiment, as illustrated in FIG. 1B. In reflective embodiments, a reflection accessory 108 may optionally be included to system 100 to efficiently redirect one or both input light 103 and output light 106 to and from the sample 104 while maintaining optimum sample positioning. It is within the scope of the disclosure for the system 100 to include a processor 109 to perform the classification model 201, including process, analyze, and classify the spectrum 107. The processor 109 may be directly connected to the detector 102 or be removed in physical space and process time.

### Optical Mechanism

The present disclosure utilizes an optical mechanism 101, as illustrated in FIGS. 1A and 1B, to produce wavelengths of light that are directed onto a sample 104. The optical mechanism 101 may comprise any suitable light generating system as known in the art. In some of the currently preferred embodiments, the optical mechanism 101 utilizes radiation in the mid-infrared wavelengths. In one currently preferred embodiment, the optical mechanism 101 is a quantum cascade laser (QCL) capable of producing mid-infrared (MIR) wavelengths of light, the wavelengths typically ranging from 4.8 to 11 micrometers. Light emitted from the optical mechanism 101 is referred herein as “input light” 103. The MIR quantum cascade laser in the currently preferred embodiment is advantageously compact, widely tunable and may be high power. Preferably, the optical mechanism 101 produces lased input light 103 (i.e. light produced from a laser). In one embodiment, the optical mechanism 101 comprises a Quanta-Ray Nd:YAG pump laser equipped with a MIR generation unit. In other embodiments, the optical mechanism 101 comprises a lead salt laser, or a doped insulator laser.

QCLs are unipolar lasers that comprise layers of semiconductor material, allowing for electrons to transition to a lower energy state and release photons at precise locations. In contrast to typical diode lasers that have population inversion between the conduction and the valence band, the QCL uses intersubband transitions to achieve population inversion. Voltage is applied over a series of nanometer thin layers of various semiconductors, and the electron transitions between the quantum wells created by the voltage creates laser emission. Since the wavelength of the output light is determined by the structure of the semiconductor layers and not the material, an advantage of QCLs is that they are tunable across the mid-infrared spectrum from 5.5 to 11.0 µm (900 cm-1 to 1800 cm-1). QCLs emit photons at every intersubband transition, while a typical diode laser would release one photon over the equivalent cycle, meaning that QCLs hold significantly more power. These small, powerful, tunable lasers are able to be operated at room temperature, making them very useful in the field of spectroscopy.

The optical mechanism 101 is further not directly connected to nor in contact with the sample 104, as is true for some investigation systems as known in the art. A space exists between the optical mechanism 101 and the sample 104 (or sample holder 105, if present). Preferably, only the input light 103 travels through the space. The optical mechanism 101 directs input light 103 through the light path 113 towards the sample 104. Because the sample 104 does not physically touch the optical mechanism 101, cross contamination does not occur in the present system 100. The only components likely to be touched by the sample 104 is the sample holder 105. Typically, these components are configured to be exchangeable or expendable (i.e. single use or easily removable and washable). Preferably, any component that touches the sample 104 will not become contaminated by the sample 104 at the light path 113. For example, the sample holder 105 does not contact the sample 104 at the points that input light 103 and output light 106 is received and generated, respectively.

### Sample

The present disclosure accepts a sample 104 to be investigated. Typically, the sample 104 is predefined as a potential plastic sample or a sample potentially containing plastic by a separate system before investigation with an embodiment of the present disclosure. Once selected, the sample 104 is placed such that the input light 103 may be directed onto the sample 104. An optional sample holder 105 may be used to hold sample 104 in the path of the input light 103; sample holder 105 may be any suitable mechanism to retain the sample 104 during investigation. Typically, the sample holder 105 is a physical piece that the sample 104 rests on. In the currently preferred embodiment, a sample optimizer 110 is further used to place the sample 104 in the best condition for investigation, comprising a mirror 111 opposite to the direction of directed input light 103 from the optical mechanism 101 and a restrainer 112 maintaining the mirror’s 111 placement, as well as the sample’s 104 placement. In some embodiments, the restrainer 112 is a suitable weight block placed on top of the mirror 111. In the currently preferred embodiment the weight is 1 kg or less. In another embodiment, the restrainer 112 is a lockable holder holding both the mirror 111 and sample 104. In other embodiments, the restrainer 112 is a force that holds the mirror 111, for example air or magnetic force restraining the mirror 111 and the sample 104 in a suitable place.

For the sample holder 105 to be suitable, the sample holder 105 must present the sample 104 to the input light 103 such that the sample 104 and input light 103 will produce output light 106, as described elsewhere herein. The placement of the sample 104 and sample holder 105 will depend on the embodiment. In the currently preferred embodiment, the sample 104 is substantially in a dry state and not suspended in a liquid. The sample 104 may originate from a liquid sample and be collected for presentation on the sample holder 105, for example, restrained on a filter or strainer, and then dried sufficiently.

Sample 104 arrangement in light path 113. The path taken by the input light 103 and output light 106 is referred herein as the “light path” 113 and the light path 113 may have different trajectories in different embodiments. In one embodiment, the light path 113 of system 100a is a direct line from the optical mechanism 101, through sample 104 and to the detector 102, as illustrated in FIG. 1A. In this embodiment the light path 113 is set up to transmit through the sample 104. Transmission works best with thin samples 104; additionally, the sample holder 105 and weight may be optically transparent to best enable transmission. In some embodiments, the light path 113 transmits through the sample 104, but includes one or more reflection accessories 108 to redirect the light path 113.

In other embodiments the system 100b reflects light from the sample 104 (as illustrated in FIG. 1B) and the light path 113 is thus changed after the sample 104. In some of these refection embodiments, only the sample 104 reflects the light path 113 and the light path 113 only spans from the optical mechanism 101, sample 104, and detector 102. In other refection embodiments, the light path 113 spans from the optical mechanism 101, a reflection accessory 108, sample 104, back to the same reflection accessory 108, and the detector 102. The output light 106 may be thought of being created when it exits or reflects off the sample 104. In transmissive embodiments, the input light transmits through the sample once and does not re-enter the sample. In some reflective embodiments, the input light transmits through the sample a first time, reflects off a mirror 111 and transmits a through the sample a second time. In some reflective embodiments, the output light 106 reflects at an angle parallel to the input light 103. In other reflective embodiments, the output light 106 reflects at an angle other than 180 degrees from the input light 103 (i.e., not in the same direction as the input light 103 was directed).

### Reflection Accessory

To optimize sample investigation, the system 100 often comprises a reflection accessory 108. The reflection accessory 108 is optional, and input light 103 may be directed directly from the optical mechanism 101 to the sample 104. However, the reflection accessory 108 enables simplified and often easier sample placement while not negatively affecting sample investigation. The reflection accessory 108 redirects input light 103 to a sample 104 in a sample holder 105 as well as redirects output light 106 from the sample 104. In the currently preferred embodiment, a spectral reflection accessory 108 redirects, by 45 degrees in one embodiment, input light 103 towards a sample 104 secured in a sample holder 105.

It is within the scope of this disclosure to provide both a reflective system 100b or a transmissive system 100a. In some embodiments, light transmits through the sample 104, in other words, the output light 106 is 180 degrees opposite from the input light 103. In some embodiments, the light reflects at an angle from the sample 104 (see FIG. 1B). In some reflective embodiments, opposite the sample 104 (from the reflection accessory 108) is a mirror 111, which causes output light 106 to re-enter the reflection accessory 108 and is redirected again at -45 degrees (see FIG. 1B) to a detector 102. In less preferred embodiments, output light 106 is collected (i.e. captured by a detector 102) both by reflection and transmission; these embodiments preferably comprise two detectors 102.

In one embodiment, a reflection accessory 108 reflects the output light 106 in a parallel, but opposite direction as input light 103, enabling the optical mechanism 101 to be in close proximity to detector 102. Additional mirrors may be present in this and other embodiments to adjust the light path, as known in the art.

### Detector

The present disclosure provides a detecting mechanism for the capture and detection of output light 106 from a sample 104, referred herein as detector 102. The detector 102 may be any suitable detecting mechanism as known in the art. In the currently preferred embodiment, the detector 102 is a thermoelectrically-cooled mercury-cadmium-telluride (MCT) detector 102 commercially available from Vigo Systems S.A. coupled to a pre-amplifier 115.

The output light 106 may further be modified before being received by the detector 102. In some embodiments, light modifiers 114 (e.g. polarizers) are placed within the light path 113 to reduce or modulate light in the light path 113. The light modifiers 114 may be placed to affect the input light 103, as illustrated in FIG. 1A with light modifiers 114a; or be placed to affect the output light 106, as illustrated in FIG. 1B with light modifiers 114b. In either case, light received by detector 102 is affected by light modifiers 114. Detectors 102 may be prone to saturation, causing flattening of spectral peaks or other artificial appearances of spectral features. Light modifiers 114 may fix saturation or other issues, depending on the specific embodiment. In the currently preferred embodiment, a pair of CaF2 polarizers 114b reduce the output light 106 levels without significantly affecting the spectrum 107.

The detector 102 may optionally be coupled to an amplifier 115 that increases the power of signal of the detected spectrum 107. The amplifier 115 may be any suitable mechanism as known in the art. In some embodiments, the amplifier 115 is a separate device interconnected to the detector 102. In other embodiments the amplifier 115 is incorporated into the detector 102. In the currently preferred embodiment, the amplifier 115 comprises a lock-in amplifier enabling the extraction of the desired spectrum 107 from background noise. Typically, the spectrum 107 from the detector 102 and amplifier 115, if present, is recorded by a digital control device 116 (e.g. a computer) or on a physical memory device 117.

### Processing Hardware

In some embodiments, system 100 may include a processor 109 to support operations and calculations of the embodiment. In a preferred embodiment, processor 109 provides a solution for processing, analyzing and classifying spectrum 107. In some embodiments, processor 109 is connected directly to detector 102. In other embodiments, processor 109 is disconnected from processor 109, instead processor 109 is connected to a memory device 117 or another control device 116, either of which is typically portable and may be delivered to processor 109. Delivery of memory device 117 or control device 116 may be physical, or by data link (i.e., data delivered wirelessly with a solution like WiFi).

Processor 109 may be any suitable solution as known in the art. The processor 109 may further provide instructions to other components in system 100. In some embodiments, processor 109 enables real-time operational control of system 100. In some embodiments, processor 109 may consist of a central processing unit (cpu), random access memory (RAM), long-term memory storage, and optional analog to digital converters. In some embodiments, processor 109 implements a user interface.

### Spectrum Processing

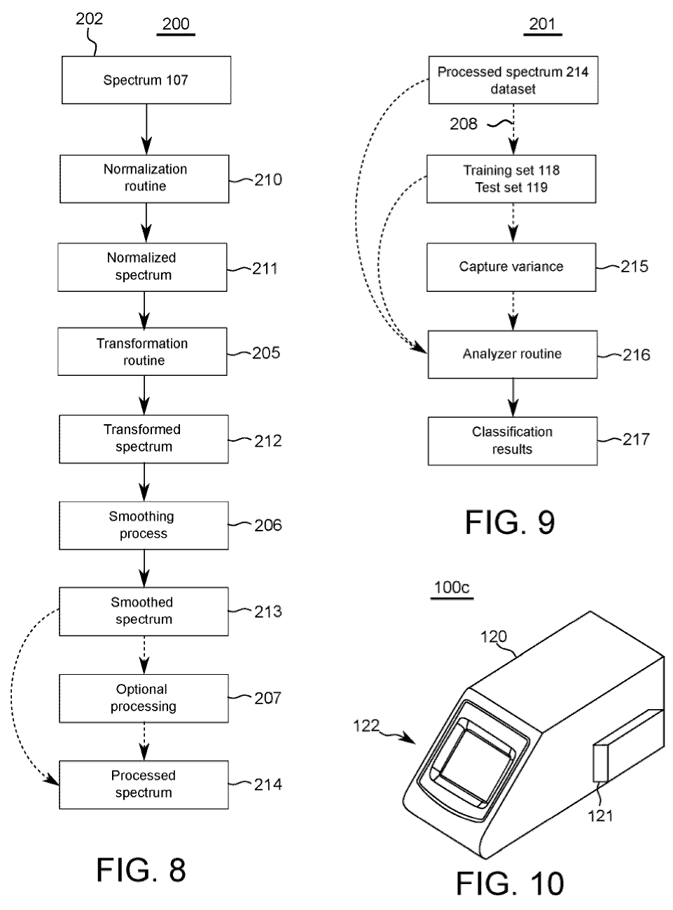

The outputted spectrum 107 from system 100 may further be processed according to the present disclosure and one possible processing method 200 is illustrated in FIG. 8. In one embodiment, a normalization routine 210 is applied to spectrum 107 from each sample I(v) by dividing by the background mirror spectrum I₀(v) (step 202 in FIG. 8), and then converted to the normalized specular absorbance spectrum according to Equation #1, resulting in a normalized spectrum 211.

$$A \left(v\right) = \text{-log} \left(I \left(v\right) / {I}_{0} \left(v\right)\right) . \qquad \text{Eq. \#1:}$$

The resulting normalized spectrum 211 is then converted to a transformed spectrum 212 by a transformation routine 205. In one preferred embodiment, the normalized spectrum 211 is transformed into the normalized spectrum’s imaginary analytic signal, using a suitable transformation routine 205. The transformation routine 205 may be, for example, Hilbert transform, Kramers-Kronig, and the like, or a combination of more than one of these transformation routines. The resulting transformed spectrum 212 may be smoothed using a smoothing routine 206, resulting in a smoothed spectrum 213. Additional optional processing 207 may be performed in addition to the above if needed. Optional processing 207 may include any solution as commonly known in the art. Typically, additional, optional processing 207 results in noise reduction. One example of optional processing 207 includes removal of a region of spectrum 107; see Example below. The end result of the method 200 is a processed spectrum 214, produced typically by the smoothing routine or the optional processing 207. If, in a given embodiment, there is no optional processing 207, then the smooth spectrum 213 is the same as the processed spectrum 214. Optional steps are illustrated in FIGS. 8 and 9 by dotted arrows.

### Classification Model

After spectrum processing and transformation in method 200 above, a classification model 201 enables classification of sample type, as illustrated in FIG. 9. Typically, the classification model 201 will classify at least one property of the spectrum applied to the model. It is within the scope of the present disclosure to apply spectra with differently applied processing steps to the classification model. In some embodiments, a dataset containing a plurality of sample spectra may be split into training set 118 and test set 119. For the purpose of this discourse, the terms “dataset” and “set” are interchangeable. Typically, the dataset of spectra is a set of processed spectrum 214 as described above. However, it is within the scope of the present disclosure for the dataset to be spectrum 107 without spectrum processing.

In some cases, a training set 118 may be obtained previously or otherwise separately from a test dataset 119. If the dataset is split, any suitable splitting routine 208 may be used, for example a stratified random splitting, to ensure the prediction accuracy on each class was equally weighted in the test accuracy. Additionally, a data variance control routine 215 may be implemented to reduce variance by any suitable process, for example random resampling and re-analysis of the dataset.

Next an analyzer routine 216 is applied to the output of the above steps; either to the processed spectrum 214, the training set 118, the test set 119, or a combination thereof. In addition, the data variance control routine 215 may be performed on the one or more datasets (i.e., training set 118 and test set 119) and the results may be taken into consideration by the analyzer routine (each optional step being illustrated by a dotted arrow in FIG. 9). The analyzer routine 216 may be any suitable solution as known in the art. The analyzer routine 216 enables the characterization, classification, or other separation of two or more classes of objects or events; here spectra of interest into sample types (e.g., chemical composition of a sample 104). In some embodiments, the analyzer routine 216 is a statistical combination method, for example a linear combination as known in the art. In some embodiments, the analyzer routine 216 is a linear discriminant analysis, a technique used to reduce the number of variables in a dataset that can be used as a classifier for modeling differences of groups. In other embodiments, the analyzer routine 216 is an analysis of variance (ANOVA); in still other embodiments, the analyzer routine 216 is a regression analysis. The analyzer routine 216 results in a classification of the spectra subjected to that analyzer routine, resulting in the production of classification results 217.

The analyzer routine 216 is preferably applied to only the spectra of interest (i.e., test set 119). Alternatively, the analyzer routine 216 may be applied to both training set 118 and test set 119. Typically, the analyzer routine 216 compares small differences in spectral peaks between two or more spectra in the datasets to determine if classification results 217 was correct. In some cases, entire spectra are compared, in other cases a subsection, or region, is compared. Spectral peaks are defined as peak position in wavelength. Some embodiments may use power spectral density, peak height, peak area, left width, right width, left boundary, right boundary, or a combination thereof for comparison. In some cases, a power spectral density of the spectra is calculated and compared. Power spectral density describes the distribution of power into frequency components composing that signal (i.e., of the spectra). Power spectral density may apply to the entire spectra or to only portions of the spectra. Summation or integration of the spectral components yields the total power (for a physical process) or variance (in a statistical process).

The present disclosure provides for a classification model 201 to classify sample-derived spectra 107 into different types of plastics. For the purposes of this disclosure the terms class, type, category, and group are used interchangeably. The classification model 201 may be any suitable model as known in the art. Typically, the classification model 201 will assign a classifier algorithm that maps spectra into specific categories. In some embodiments, the classification model 201 maps a portion of a spectra into a category. In other embodiments, the classification model 201 maps an entire spectra into a category. Features in subregions may be assigned from known plastic type spectra for identification purposes. Alternatively, or in addition to, training sets 118 of spectra from known plastic type may be used for identification purposes. Classification may be multi-class; for example, assigning one of many plastic classes to a spectrum 107. Classification may also be multi-label; assigning, for example, a plastic class as well as another classification (e.g. plasticizer class) to a spectrum 107.

### Field Portable

Some embodiments of the present disclosure are further field portable and illustrated in FIG. 10. These portable embodiments are of such size and weight as to be conveniently portable. These portable embodiments are encased in a housing 120 enabling protection from environmental factors including water, corrosion, temperature, dust, sand, light, and the like. Housing 120 at least partially encloses the majority of components described in system 100 above. Housing 120 further accommodates a power source 121 (e.g., a battery) to provide power when system 100c is in use. Preferably the power source 121 is rechargeable and preferably rated at 12 Volts or less. In some embodiments, the power source 121 is between 12 and 16 Volts.

Preferably the processor 109 is adapted to connect to an interface 122 and is also adapted to prevent the supply of power from power source 121 to at least the optical mechanism 101 when the system 100c has not been operated for a predetermined time period. Interface 122 may be any suitable solution as known, in a currently preferred embodiment, interface 122 is a graphical user interface. Interface 122 may comprise a screen for outputting operational settings and performance characteristics to a user. Preferably the screen is a touch screen adapted to allow the user to input or modify various operational settings. The interface may further comprise a speaker and a speaker driver circuit. The interface, along with the processor 109, may be adapted to transmit a signal to the optical mechanism 101 when one or more operational conditions exceed a predetermined value. Portable embodiments may be manually picked up by a user and moved by a user’s force alone (e.g., no motorized assistance used).

### EXAMPLE

One specific embodiment of the instant disclosure is described in detail presently. This embodiment of system 100b is illustrated in FIG. 1B and method 200 results in spectra 107 illustrated in FIG. 2A-6. A system 100b having a sample holder 105, an optical mechanism 101, a detector 102, and a processor 109 was established to investigate samples 104 from purchased consumer, laboratory, and hardware products with plastic type identified based on imprinted recycling code labels (see Table 1). Thirty samples 104 of each of five types of plastics, PET, HDPE, LDPE, PP, and PS, for a total of 150 samples 104 were selected. The plastic samples 104 included a range of color, opaqueness, and thickness. Thin film plastics were not selected due to the challenge they present with interference fringes from back-surface reflection; thus, all samples 104 selected were at least 0.13 mm thick. All samples 104 were rinsed with deionized water and cut to a size of approximately 2 cm × 2 cm before analysis on the system 100b.

TABLE 1

Thirty consumer plastic samples from five different types of plastics were selected^{a}.

| Recycling Code | Plastic Type | Sample Colors | Sample Opaqueness | Thickness Range (mm) |
|---|---|---|---|---|
| 1 | Polyethylene terephthalate (PET) | Clear, red, blue, green | Clear, opaque | 0.13 – 1.62 |
| 2 | High density polyethylene (HDPE) | White, green, black, orange, brown, clear, turquoise | Semi-opaque, opaque | 0.42 – 2.02 |
| 4 | Low density polyethylene (LDPE) | Clear, white, red, blue, yellow | Clear, semi-opaque, opaque | 0.59 – 12.74 |
| 5 | Polypropylene (PP) | Black, clear, white, blue | Clear, semi-opaque, opaque | 0.42 – 1.36 |
| 6 | Polystyrene (PS) | White, purple, clear, black | Clear, semi-opaque, opaque | 0.20 – 11.57 |

^{a}The samples selected included a range of colors, opaqueness, and thickness.

The system 100b comprised a widely tunable (5.59 - 7.41 µm / 1789.871350.07 cm⁻¹) optical mechanism 101 of a pulsed external cavity QCL (maximum average power 28 mW; Daylight Solutions Inc.). This optical mechanism 101 was selected based on its wavelength coverage of key spectral peaks, identified previously by ATR-FTIR, of the five targeted plastics. The QCL was pulsed at a 5.0% duty cycle, 100 kHz pulse repetition rate, with a 500 ns pulse width. A 45-degree fixed angle specular reflection accessory 108 (Pike Technologies, 45Spec Accessory, 011-4500) with a 10 mm mask was utilized for sample analysis. Reflection accessory 108 was incorporated into sample holder 105 for this embodiment, however they may be separate in other embodiments. Samples 104 were laid across the opening of the mask. A sample optimizer 110 comprising a gold mirror 111 followed by a weight were placed on top of each sample 104 to maintain or improve sample 104 flatness as the plastic samples 104 were often irregular in thickness. A 9 µm thermoelectrically-cooled mercury-cadmium-telluride (MCT) detector 102 (Vigo -PCI-2TE-9) coupled to a pre-amplifier was used for collection of specularly reflected light (i.e., spectrum 107). Two CaF₂ holographic wire grid polarizers 114 (Thorlabs, WP25H-C) were placed in the beam path to reduce the amount of light received by the detector 102 to avoid saturation. A Zurich Instruments - HF2LI lock-in amplifier 115 was used for signal collection from the detector 102 and data were recorded using MATLAB. A background measurement using the gold mirror 111 was collected prior to the measurement of every fifth plastic sample 104, to monitor any changes in QCL output power. For each mirror 111 or plastic sample 104 measurement, the QCL was scanned across its full tuning range 5 times. Each output spectrum 107 recorded was thus the average of 5 spectra. Each plastic sample 104 was analyzed in triplicate, moving the sample 104 between each measurement; resulting in 450 total spectra (three reflection spectra of each of the 180 plastic samples 104).

Processing. In this example, the QCL spectrum 107 of each plastic sample 104 was processed as described above. Normalized spectrum 211 (generated using Equation #1) was transformed using a transformation routine 205 comprising the Hilbert transform function in MATLAB, as an alternative to the Kramers-Kronig. A smoothing routine 206 using a moving average of 150 was used to smooth the transformed spectrum 212, and further optional processing 207 was the removal of the 1697-1550 cm⁻¹ region from each transformed spectrum 212 due to a lack of identifying spectral features for plastics in this region, resulting in a processed spectrum 214. The removal of this region enabled a reduction in size of the spectral dataset and limited excess noise from entering the classification model 201.

For the classification model 201, linear discriminant analysis was implemented using the MATLAB Machine Learning toolbox and was utilized to develop the classification model 201. The dataset of 450 total spectra was split using a splitting routine 208 into a training set 118 (two-thirds of the samples 104) and a holdout test set 119 (remaining third of the samples 104). The holdout test set 119 was chosen (i.e., by splitting routine 208) via a stratified random sample to ensure that the prediction accuracy on each class was equally weighted in the test accuracy. In order to capture any variance in the data, the training set 118 and test set 119 may be subjected to data variance control routine 215 comprising the randomly resampling of ten times and the analysis was repeated as separate trials. The final reported test accuracy (classification results 217) and confusion matrix results are the average of the ten repeated trials.

Linear discriminant analysis was then applied to only the HDPE and LDPE spectra to confirm that these plastic types could be classified correctly based on small differences in their two spectral peaks in the 1477-1458 cm-1 region. To confirm this, only HDPE and LDPE were used in a second classification model 201 following the same approach as when all the plastics were used. Two approaches were used, first, using the full spectral region (minus the omitted portion as described previously) and, second, using only the 1477-1458 cm-1 region of the HDPE and LDPE samples 104.

Attenuated total reflectance-Fourier transform infrared (ATR-FTIR) spectroscopy and Fourier transform infrared (FTIR) spectroscopy were used to confirm the above findings with the presently exemplified system 100b. Five representative samples 104 were selected for ATR-FTIR analysis (PET02, PP22, HDPE28, LDPE17, PS05) to determine peak position for comparison with the QCL-reflectance data. ATR-FTIR was performed on these five samples 104 in triplicate. These measurements were made using an Agilent Technologies Cary 630 FTIR spectrometer coupled to a D-ATR diamond crystal accessory with a single reflection sensor and a sample press. Absorbance spectra were collected using 32 scans at a 2 cm-1 resolution measuring between 4000 - 650 cm⁻¹. A background atmospheric spectrum was subtracted from all sample spectra.

A Bruker Vertex 80 Fourier transform infrared spectrometer was utilized in reflectance mode using the 45-degree fixed angle specular reflection accessory (Pike Technologies, 45Spec Accessory, 011-4500) in the sample compartment. A broadband mid-infrared globar along with a KBr beamsplitter, and a liquid nitrogen-cooled MCT detector with a ZnSe window that covers the 12,000 cm⁻¹ to 600 cm⁻¹ region. Each plastic was placed on top of the reflection accessory and a gold mirror was used to calculate a background spectrum. Spectra were collected using 32 scans with a spectral resolution of 2 cm⁻¹ measuring between 1300 - 1800 cm⁻¹. The Hilbert transform was applied using MATLAB, and spectra were smoothed with a moving average of 10.

The presently exemplified system 100b produced QCL reflectance spectra that showed clear peaks corresponding to known distinct features for all five of the plastic types, with similarities in peak location for HDPE and LDPE (see FIGS. 2B and 2D, respectively). Significant spectra-to-spectra variability among replicate runs of the same sample 104 existed, which may be attributed to changes in reflection due to how the sample 104 was placed on the reflection accessory 108, as each sample 104 was moved between replicates. However, the key spectral features did not vary in location. Variability existed between different samples 104 of the same plastic type, which may be attributed to variations in plastic formulation (e.g. stabilizers, fillers, colorants, and additives) and physical variability of the samples 104 (e.g. differences in smoothness, shininess, opaqueness, and color) (see FIG. 3). Interference fringes patterns were observed in some spectra. Despite the differences between spectra of the same plastic type, the distinct spectral features identified for each type of plastic appeared in 95% of all spectra. Only in 21 measurements out of the 450 measurements were spectral features not clearly identifiable.

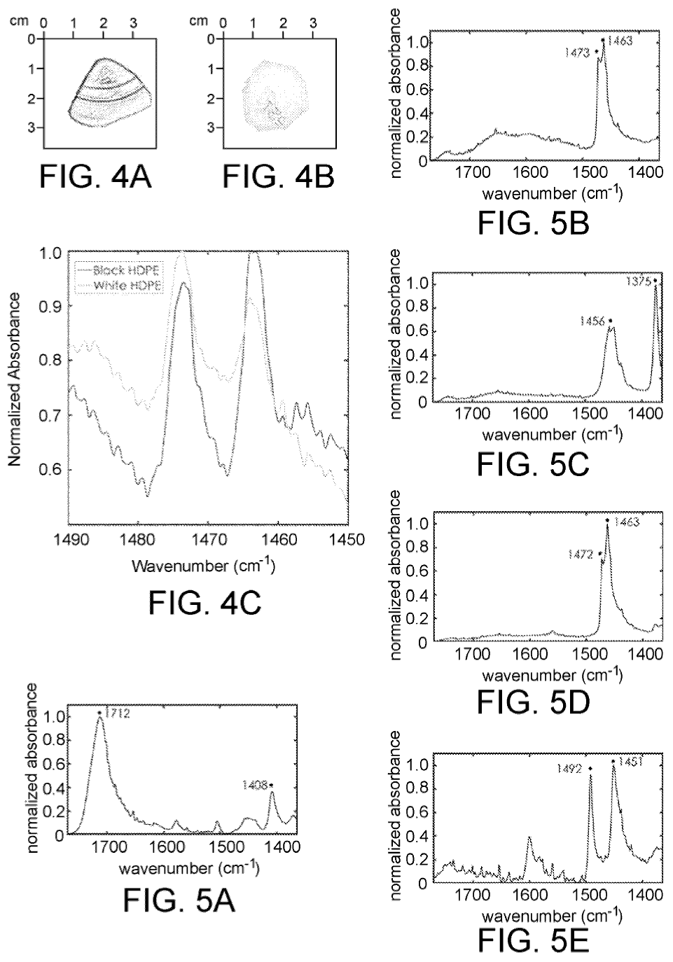

To examine the influence of color on spectra, a black HDPE sample 104 was compared to a white HDPE sample 104 analyzed using the presently exemplified QCL reflectance setup. The spectra show clearly visible peaks at 1473 cm-1 and 1463 cm-1 in both spectra and illustrated in FIGS. 4A-4C. This ability to analyze dark plastic samples 104 is an advantage of the utilization of mid-infrared wavelengths instead of near-infrared for plastics identification.

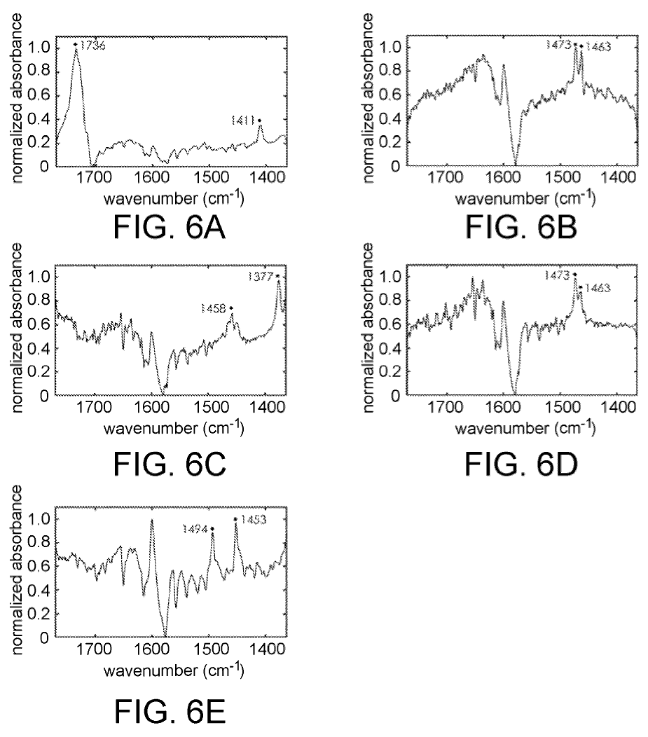

Comparisons of peak locations and ease of analysis for QCL, ATR-FTIR and FTIR. The spectral peaks present in the QCL reflectance spectra were compared to ATR-FTIR spectra reported in the literature as well as spectra taken in the laboratory using both ATR-FTIR (see FIGS. 5A-E) and FTIR-reflectance spectroscopy (FIGS. 6A-6E, Table 2) set-ups described in this example above. Limitations in these techniques must be noted as ATR-FTIR requires that the sample 104 be physically in contact for the measurement and the FTIR measurements required the use of a liquid nitrogen-cooled detector. For ATR-FTIR, the peak locations reported in the literature aligned with those measured presently (see FIGS. 5A-5E; Table 2). ATR-FTIR is routinely used for plastics analysis including microplastics analysis. Differences in peak location however were observed between the ATR-FTIR measurements when compared to those seen in the FTIR-reflectance and QCL-reflectance data, both of which agreed with each other. At the longer wavelengths measured, ATR spectral peaks are often shifted towards lower frequencies (shift in peak position) when compared to transmission or reflectance spectra. Since plastics are routinely identified spectrally in the infrared region by their characteristic peaks, it is important to recognize these shifts.

TABLE 2

Spectral peaks^{a}

| Plastic Type | ATR Peak (cm⁻¹) (previously reported) | ATR Peak (cm⁻¹) Laboratory | Reflectance Peak - FTIR (cm⁻¹) | Reflectance Peak QCL (cm⁻¹) |
|---|---|---|---|---|
| PET | 1713 C=O stretch | 1712 | 1736 | 1736 |
| | 1408 Aromatic Ring Stretch | 1408 | 1411 | 1411 |
| HDPE | 1472 CH₃ Bend | 1473 | 1473 | 1473 |
| | 1462 CH₂ Bend | 1463 | 1463 | 1463 |
| LDPE | 1467 CH₂ Bend | 1472 | 1473 | 1473 |
| | 1462 CH₂ Bend | 1463 | 1463 | 1464 |
| PP | 1455 CH₂ Bend | 1456 | 1458 | 1456 |
| | 1377 CH₃ Bend | 1375 | 1377 | 1378 |
| PS | 1492 Aromatic Ring Stretch | 1492 | 1494 | 1494 |
| | 1451 CH₂ Bend | 1451 | 1453 | 1453 |

^{a}Peaks shown have been previously reported for ATR-FTIR and were measured in the laboratory by ATR-FTIR, FTIR in reflectance mode, and using the QCL reflectance setup.

Plastic-type identification using QCL reflectance spectroscopy. The classification model 201 using linear discriminant analysis resulted in a 97% correct identification rate for the 150 samples 104 analyzed. The variability between spectra for the same plastic type is hypothesized as the cause of some misidentifications. All PET samples 104 were correctly identified (Table 3), due to the strong spectral feature at 1736 cm⁻¹. For each of the other four plastic types, the model was also highly successful, resulting in at most 9 plastic samples 104 being misidentified during a single model run, with most misclassifications occurring between HDPE and LDPE. For example, during one model run, 6 HDPE samples 104 were incorrectly classified as LDPE. HDPE and LDPE both have a spectral peak at 1463 cm⁻¹ and a closely spaced second peak at 1473 cm⁻¹ and 1472 cm⁻¹ for HDPE and LDPE, respectively. To confirm that the slight peak difference allows for the discrimination of HDPE and LDPE, linear discriminant analysis was then run on only HDPE and LDPE. When the full spectral region (minus the chopped portion as described previously) was included, the success rate for identification between HDPE and LDPE was 88 +/- 4% (Table 4). When only the peak region (1477 - 1458 cm⁻¹) was utilized, the success rate increased to 97 +/-3 % (Table 5). Therefore, this suggests that the small difference in the HDPE and LDPE peaks allows for the discrimination to take place.

TABLE 3

Confusion matrix of linear discriminant analysis for the five plastics.
Prediction

| | | PET | HDPE | LDPE | PP | PS |
|---|---|---|---|---|---|---|
| Truth | PET | 30 ± 0 | 0 ± 0 | 0 ± 0 | 0 ± 0 | 0 ± 0 |
| | HDPE | 0 ± 0 | 28 ± 2 | 2 ± 2 | 1 ± 1 | 0 ± 0 |
| | LDPE | 0 ± 0 | 1 ± 1 | 29 ± 1 | 0 ± 1 | 0 ± 0 |
| | PP | 0 ± 1 | 0 ± 1 | 0 ± 0 | 29 ± 1 | 0 ± 1 |
| | PS | 0 ± 0 | 0 ± 1 | 0 ± 0 | 0 ± 1 | 29 ± 1 |

Values on the diagonal (shaded and bolded) are correctly identified samples^{a}.
^{a}Values are the average and standard deviation of 10 repeated random splits of the data using test sets containing 30 measurements of each plastic type.

TABLE 4

Confusion matrices of linear discriminant analysis for HDPE and LDPE using full spectral region^{a}.
Prediction

| Truth | | HDPE | LDPE |
|---|---|---|---|
| | HDPE | 24 ± 3 | 6 ± 3 |
| | LDPE | 2 ± 2 | 28 ± 2 |

^{a}Values are the average and standard deviation of 10 replicates using test sets containing 30 measurements of each plastic type. Values on the diagonal (shaded and bolded) are correctly identified samples.

TABLE 5

Confusion matrices of linear discriminant analysis for HDPE and LDPE using the spectral region covering the peaks only (1477-1458 cm⁻¹)^{a}.
Prediction

| Truth | | HDPE | LDPE |
|---|---|---|---|
| | HDPE | 29 ± 2 | 2 ± 2 |
| | LDPE | 1 ± 1 | 30 ± 1 |

^{a}Values are the average and standard deviation of 10 replicates using test sets containing 30 measurements of each plastic type. Values on the diagonal (shaded and bolded) are correctly identified samples.

QCL-based MIR reflectance spectroscopy coupled to a classification model 201 using linear discriminant analysis was demonstrated to be a successful approach for rapid and robust identification of plastic type with a 97% correct identification rate. While five different types of plastics were selected that had strong spectral features in the 5.59 to 7.41 µm region, other plastics, such as polyvinylchloride (PVC), were not included in this study due to the lack of strong spectral features in this region. However, due to the ability to design and fabricate QCLs at specific wavelengths, other plastics should also be identifiable using this same approach by selecting a QCL with the appropriate wavelength region. The use of widely tunable QCLs (e.g. multiple QCLs or QCL arrays) would allow a broader range of plastic types to be distinguishable. QCL beam diameters are typically on the order of ~3 mm in diameter but can be focused down to reduce the beam to less than 300 µm and some calculations point to beam diameters achieved as small as ~20 µm. Although macroplastic samples 104 were utilized here, the use of a smaller diameter beam would make it a viable approach for the analysis of smaller (<100 µm) plastic samples 104 including microplastic (<5 mm) samples 104.

QCLs are tiny sources that can be designed to operate at mid-infrared wavelengths and at the same time can be made widely tunable. Incorporating a QCL into a small sensor, that does not require physical contact with the plastic sample 104, could have broad applications for the identification of plastic for recycling and environmental applications. If an environmental application was sought, future studies would be needed to examine plastic samples 104 collected from the environment, which have been chemically and physically weathered by environmental processes. This weathering, which could occur in both terrestrial and aqueous locations, has the potential to alter spectral peaks. The samples 104 used in this study were newly acquired plastic samples 104 and offer an important first-step in presenting the ability of QCL to identify plastic type.

Although specific features of the present disclosure are shown in some drawings and not in others, this is for convenience only, as each feature may be combined with any or all of the other features in accordance with the disclosure. While there have been shown, described, and pointed out fundamental novel features of the disclosure as applied to a preferred embodiment thereof, it will be understood that various omissions, substitutions, and changes in the form and details of the devices illustrated, and in their operation, may be made by those skilled in the art without departing from the spirit and scope of the disclosure. For example, it is expressly intended that all combinations of those elements and/or steps that perform substantially the same function, in substantially the same way, to achieve the same results be within the scope of the disclosure. Substitutions of elements from one described embodiment to another are also fully intended and contemplated. It is also to be understood that the drawings are not necessarily drawn to scale, but that they are merely conceptual in nature.

1. A system for the identification and classification of plastic type, comprising:
an optical mechanism configured to produce input light having mid-infrared wavelengths, and said input light is capable of impinging a plastic sample and whereby output light is created upon impinging the plastic sample;
a space between said optical mechanism and the plastic sample;
a detector to receive said output light, producing a spectrum; and
a processor configured to classify a first property of said spectrum using a classification model;
wherein said input light travels through said space.

2. The system of claim 1 wherein said classification model maps at least a portion of said spectrum into one category of a plurality of categories and wherein said one category of said plurality of categories is a type of plastic.

3. The system of claim 2 wherein said plurality of categories represent different types of plastic.

4. The system of claim 1 wherein said optical mechanism is a quantum cascade laser.

5. The system of claim 1 wherein said output light is created after said input light transmits a single time through the plastic sample without reflection.

6. The system of claim 1 further comprising a mirror; and wherein said output light transmits a first time through the plastic sample, reflects off said mirror and transmits a second time through the plastic sample.

7. The system of claim 1 further comprising a housing, an interface, and a power source; said housing provides protection from environmental; and wherein said optical mechanism, said space, said detector, said processor, and said power source are contained within said housing.

8. The system of claim 1 further comprising a light modifier configured to modify the output light.

9. The system of claim 1 wherein said classification model is further configured to normalize and transform said spectrum.

10. The system of claim 9 wherein said classification model is supplied with a training set and a test set, and said classification model is further configured to apply an analyzer routine on at least said test set.

11. A method to classify a type of plastic, the method comprising the steps of:
(a) selecting a system having an optical mechanism, a sample holder, a space between said sample holder and said optical mechanism, and a detector, said optical mechanism producing input light suitable for impinging a sample, said input light consisting of mid-infrared wavelengths;
(b) placing a plastic sample onto said sample holder;
(c) directing said input light across said space and onto said plastic sample, producing output light;
(d) capturing said output light by said detector, producing a spectrum; and
(e) analyzing said spectrum with a classification model to classify at least one property of said spectrum, producing a classification result.

12. The method of claim 11 wherein said classification model classifies said at least one property of said spectrum into one category of a plurality of categories; and wherein said one category of said plurality of categories is a type of plastic.

13. The method of claim 12 further comprising the steps of:
(f) applying a normalization routine to said spectrum, producing a normalized spectrum; and
(g) applying a transformation routine to said normalized spectrum, producing a transformed spectrum;
wherein said spectrum in step (e) is said transformed spectrum.

14. The method of claim 12 further comprising the steps of:
(f) applying a normalization routine to said spectrum, producing a normalized spectrum;
(g) applying a transformation routine to said normalized spectrum, producing a transformed spectrum; and
(h) applying a smoothing routine to said transformed spectrum, producing a smoothed spectrum;
wherein said spectrum in step (e) is said smoothed spectrum.

15. The method of claim 14 further comprising the steps of:
(i) repeating steps (b) though (h); thereby producing a plurality of smoothed spectrum;
(j) forming a test set from said plurality of smoothed spectrum; and
(k) applying an analyzer routine to said test set to produce, in part, said classification result.

16. The method of claim 11 wherein said output light is created after said input light transmits a single time through said plastic sample without reflection in step (c).

17. The method of claim 11 wherein said system further comprising a mirror; and wherein in step (c), said output light transmits a first time through said plastic sample, reflects off said mirror and transmits a second time through said plastic sample.

18. The method of claim 11 wherein said system further comprising a mirror; and wherein in step (c), said output light transmits a first time through said plastic sample and reflects off said mirror at an angle other than 180 degrees from said plastic sample.

19. The method of claim 11 wherein said system further comprises a housing, an interface, and a power source; said housing provides protection from environmental factors; and wherein said optical mechanism, said space, said detector, said processor, and said power source are contained within said housing.

20. The method of claim 19 further comprising the step of (1) moving said system by a user.
